(19) United States
(12) Patent Application Publication
(10) Pub. No.: US 2010/0152828 A1
(43) Pub. Date: Jun. 17, 2010

(54) DEVICES AND METHODS FOR ACCESSING AND TREATING AN ANEURYSM

(76) Inventors: R. Sean Pakbaz, San Diego, CA (US); Charles W.. Kerber, La Mesa, CA (US)

Correspondence Address: GREENBERG TRAURIG, LLP (SV);IP DOCKETING, 2450 COLORADO AVENUE, SUITE 400E, SANTA MONICA, CA, 90404, US

(21) Appl. No.: 12/513,298

(22) PCT Filed: Nov. 2, 2007
(86) PCT No.: PCT/US07/83505

§ 371 Date: Dec. 4, 2009

Related U.S. Application Data
(60) Provisional application No. 60/864,013, filed on Nov. 2, 2006.

(51) Int. Cl. A61F 2/82

(52) U.S. Cl. 623/1.11; 623/1.15

(57) ABSTRACT

Devices for treating aneurysms are disclosed. The devices are adapted and configured to modify blood flow at the aneurysm. More specifically, the invention discloses devices for treating cerebral aneurysms using devices adapted and configured to be delivered to a blood vessel in the brain on a distal tip of a microcatheter. The aneurysm devices comprise: a device adapted to be delivered to a blood vessel aneurysm on a distal tip of a catheter and further adapted to modify blood flow at the aneurysm.

### CROSS-REFERENCE

This application also claims the benefit of U.S. Provisional Application No. 60/864,013, filed Nov. 2, 2006 by R. Sean Pakbaz et al. entitled Complex Curve Microcatheters for Berry Aneurysm Endovascular Therapy, which is incorporated herein by reference in its entirety.

### BACKGROUND OF THE INVENTION

An aneurysm is an abnormal widening or ballooning of a portion of an artery, related to weakness in the wall of the artery or blood vessel. Some common locations for aneurysms include: the aorta; the brain (cerebral); the legs (popliteal artery aneurysm); the intestine (mesenteric artery); and the spleen. Aneurysms are either congenital (present before birth) or acquired. It is thought that defects in some component(s) of the artery wall may be responsible for aneurysms. Although in some instances, high blood pressure is thought to be a contributing factor. Atherosclerotic disease (cholesterol buildup in arteries) may also contribute to the formation of certain types of aneurysms. As a result of a defect in the artery wall, the aneurysm can rupture, which can result in profuse bleeding.

Early investigators from the 18^{th} century began experimenting with procedures that involved inserting needles into an aneurysm to induce thrombosis. As a result of a lack of the imaging modalities available today, small or intracranial aneurysms could not be detected and only large lesions of the thoracic or abdominal aorta (as well as proximal extracranial carotid artery and limb aneurysms) could be identified. At the time, unpredictable results were observed and the practice of inducing thrombosis was abandoned.

Following the early failures of attempts to induce aneurysm thrombosis with percutaneous needle insertion led to a renewed interest in medical interventions. One of the earliest medical interventions was the use of potassium iodide for syphilitic aneurysms and aneurysm-related pain. The mechanism of action of potassium chloride was thought to be related to the reduction in the pulse and blood pressure, which in turn led to thrombosis. Other medications included ablation with vinegar, iron perchloride, alcohol, zinc chloride, gelatin, sodium chloride, or ergot salts. These remedies failed to find widespread application due to lack of sound scientific evidence and were soon abandoned because of inconsistent effects.

A clinical observation of a fibrin-coated bullet recovered from an autopsy case, resulted in the postulation that inserting a wire into an aneurysm would provide a much more ideal environment for clot formation, as opposed to the insertion of a simple needle. Upon autopsy it was observed that the coils of wire were filled with fibrinous material and was adherent. As this procedure found widespread application, some of the possible short- and long-term complications became evident, including an increased risk of hemorrhage from subtotal packing and distal embolization of wire or thrombus.

Over the years, advances in surgical procedures allowed for a combined approach by which an aneurysm was exposed surgically and a wire was inserted through a trocar. This approach continued in use as late as 1951. A combined approach of using a laparotomy with a trocar in a position to visualize targets was used. The aneurysm was packed through multiple sites with up to 965 feet of wire that had an abrasive surface. Failures were attributed to underpacking of the aneurysm. The early attempts and advancements made by these clinicians became the fundamentals on which current endovascular treatment of intracranial aneurysms is based. Although the initial attempts made by these pioneers yielded limited success because tools that could be used to navigate the complex intracranial vasculature and execute the treatment modality were lacking, technological advances and improvements facilitated a shift from the extravascular approach to the more physiological endovascular approach. Over time, the shift to catheterization became possible. Initially, a glass chamber was surgically connected to the patient's external carotid artery and then tubing was introduced into the internal carotid artery to access the intracranial vasculature.

Further improvements in the endovascular area led to the use of balloon-tipped microcatheters. This new direction followed the introduction of the Fogarty catheter. This device was developed for the extraction of arterial emboli and thrombi, and led to advancements in the feasibility of balloon catheters. Soon after the development of endovascular detachable balloon embolization therapy, a number of publications describing the outcome of this method in treating various cerebrovascular lesions, including intracranial aneurysms. Due to the growing experience with the detachable balloon embolization approach, a number of problems with this method also became evident.

During initial development, access to the aneurysm was challenging because a guidewire could not be used during catheterization. Second, once the lesion was finally reached, the balloon did not achieve full occlusion of the aneurysm because the device was round or oval and aneurysms take a variety of configurations. Additionally, balloons that do not fully conform to the irregular dimensions of the aneurysm sac are ineffective due in part to the pulsating arterial blood. Over time, the balloon occlusion devices may slowly deflate if they are not filled with nonsolidifying substances. Although parent artery occlusion can still be performed despite this disadvantage, balloon occlusion of the aneurysm sac has been largely abandoned in favor of more novel techniques.

The next endovascular approach designed for the selective occlusion of aneurysms was the coil. Modern metallic coils had been available for endovascular arterial occlusion and embolization since 1975, although the use of coils specifically for the treatment of intracranial aneurysms did not occur until the very late 1980s. Subsequently, the use of an endovascular coil embolization with “pushable” platinum coils was employed. One disadvantage to this method was the inability to retrieve the coil after placement. Further refinements of the coil continued, eventually leading to the development of the detachable coil. Coils have since been combined with electrolysis to achieve electrothrombosis (see, Guglielmi detachable coil, available from Boston Scientific). Although it is relatively straightforward to place the tip of a microcatheter into a cerebral berry aneurysm for detachable coil delivery, as endovascular treatment of that aneurysm progresses, it is common to find the tip of the currently used catheter being pushed out of the aneurysm, sometimes making safe or complete coil delivery into the aneurysm space impossible.

Like other aneurysms, cerebral aneurysms may occur as a congenital defect or may develop later in life. One type of cerebral aneurysm is the berry aneurysm, which can be over 2 cm in size. The berry aneurysm resembles a sack of blood attached to one side of the blood vessel and typically has a narrow neck. Other types of aneurysms involve widening or dilation of the entire circumference of a blood vessel in an area. Still other types appear as a ballooning out of a part of a blood vessel. It is estimated that 5% of the population has some type of aneurysm in the brain, with up to 10% of those affected having more than one aneurysm. The vessel wall of an aneurysm can be as thin as 15-100 microns. Cerebral aneurysms can rupture and cause bleeding or hemorrhaging in the area between the brain and the surrounding membrane (the arachnoid); or can extend into the subarachnoid space. It is generally thought that, most aneurysms under ¼ inch in diameter do not rupture. However, with currently available imaging techniques it is now possible to characterize the walls of the aneurysm and possibly predict their behavior, instead of relying on generalities. However, aneurysms that do rupture can have serious consequences including stroke and death. Approximately 20,000 people in the United States suffer a subarachnoid hemorrhage each year. An estimated 1 to 2 percent (three to six million) of Americans have cerebral aneurysms. Although they can occur at any age, they are slightly more common in adults than children and are slightly more common in women than men.

The first catheter cerebral angiograms were performed with straight, uncurved catheters. Uncurved catheters were time consuming for surgeons to use. Catheter cerebral angiography as a technique did not gain clinical acceptance until angiographers learned to place curves at the tip of the catheter in a single plane, which resulted in faster, safer and more selective studies. The next iteration was the invention and refinement of microcatheters (small catheters introduced through larger guiding catheters). These smaller catheters allowed even more selective angiography and hastened the evolution of interventional techniques.

Other devices and methods for treating aneurysms include: U.S. Pat. Nos. 5,980,514 to Kupiecki et al. for Aneurysm Closure Device Assembly; 6,096,034 to Kupiecki et al. for Aneurysm Closure Device Assembly; 6,183,495 to Lenker et al. for Wire Frame Partial Flow Obstruction Device for Aneurysm Treatment; 6,551,303 to Van Tassel et al. for Barrier Device for Ostium of Left Atrial Appendage; 6,569,190 to Whalen II et al. for Methods for Treating Aneurysms; 6,663,607 to Slaikey et al. for Bioactive Aneurysm Closure Device Assembly and Kit; 5,782,905 to Richter for Endovascular Device for Protection of Aneurysm; 5,951,599 to McCrory for Occlusion System for Endovascular Treatment of An Aneurysm; 6,063,111 to Hieshima et al. for Stent Aneurysm Treatment System and Method; 6,093,199 to Brown et al. for Intra-Luminal Device for Treatment of Body Cavities and Lumens and Method of Use; 6,168,622 to Mazzocchi for Method and Apparatus for Occluding Aneurysms; 6,626,928 to Raymond et al. for Occlusion Device for Treating Aneurysm and Use Therefore; 6,746,468 to Sepetka et al. for Devices and Methods for Treating Vascular Malformations; 6,802,851 to Jones et al. for Stent Aneurysm Embolization Method Using Collapsible Member and Embolic Coils; 6,855,153 to Saadat for Embolic Balloon; 6,860,899 to Rivelli Jr. for Method for Treating Neurovascular Aneurysms; 6,036,720 to Abrams et al. for Sheet Metal Aneurysm Neck Bridge; 6,139,654 to Teoh for Minimally Occlusive Flow Disruptor Stent for Bridging Aneurysm Necks; 5,935,148 to Villar et al. for Detachable, Varying Flexibility, Aneurysm Neck Bridge; 6,379,329 to Naglreiter et al. for Detachable Balloon Embolization Device and Method; 4,638,803 to Rand for Medical Apparatus for Inducing Scar Tissue Formation in a Body; 5,476,472 to Dormandy Jr. et al. for Embolization Device and Apparatus Including an Introducer Cartridge and A Delivery Catheter and Method for Delivering the Embolization Device; 5,746,734 to Dormandy Jr. et al. for Introducer Cartridge for Delivering an Embolization Device; 5,571,171 to Barone et al. for Method for Repairing An Artery in a Body; and US Patent Publications 2003/0018294 to Cox for Aneurysm Treatment Device and Method of Use; 2004/0044391 to Porter for Device for Closure of a Vascular Defect and Method of Treating the Same; 2004/0059407 to Escamilla et al. for Expandable Stent and Delivery System; 2004/0078071 to Escamilla et al. for Expandable Stent with Radiopaque Markers and Stent Delivery System; 2004/0111112 to Hoffman for Method and Apparatus for Retaining Embolic Material; 2004/0193206 to Gerberding et al. for Methods and Devices for the Treatment of Aneurysms; 2004/0193246 to Ferrera for Method and Apparatus for Treating Aneurysms and Other Vascular Defects; 2005/0033409 to Burke et al. for Aneurysm Treatment Device and Method of Use; 2002/0143349 to Gifford III et al. for Devices and Methods for Treating Vascular Malformations; 2002/0133190 to Horton et al. for InSitu Formable and Self-Forming Intravascular Flow Modifier (IFM), Catheter and IFM Assembly, and Method for Deployment of Same; 2002/0198592 to Wallace et al. for Intracranial Stent and Method of Use; 2003/0100945 to Yodfat et al. for Implantable Intraluminal Device and Method of Using Same in Treating Aneurysm; 2003/0109917 to Rudin for Stent Vascular Intervention Device and Method; 2003/0139802 to Wulfman et al. for Medical Device; 2003/0204244 to tiger for Aneurysm Exclusion Stent; 2005/0107823 to Leone et al. for Anchored Stent and Occlusive Device for Treatment of Aneurysms; 2005/0119684 to Guterman et al. for Aneurysm Buttress Arrangement; 2005/0133046 to Becker et al. for Compositions and Methods for Improved Occlusion of Vascular Defects; European Patent Application EP 1616585 A1 to Tijsma for Device for the Treatment of Aneurysms.

### SUMMARY OF THE INVENTION

The invention discloses devices and methods for treating aneurysms in mammals. More particularly, the invention discloses a catheter having a distal end and a proximal end and further comprising a first configuration, e.g., a configuration caused by a wire straightening the catheter into a less complex shape so it can be navigated toward or near an aneurysm, and a second configuration wherein the first configuration is adapted and configured to be delivered through a vasculature and the second configuration is adapted and configured to assume a vasculature conformable shape. The devices and methods allow a greater number of detachable coils or other embolic material to be delivered into an aneurysm by a microcatheter with less chance of displacement of the distal tip of the microcatheter from the aneurysm.

An aspect of the invention is directed to a system for accessing a cerebral aneurysm. The system comprises: a catheter having a distal end and a proximal end and further comprising a first configuration and a second configuration wherein the first configuration is adapted and configured to be delivered through a vasculature and the second configuration is adapted and configured to assume a vasculature conformable shape; and a guidewire insertable through and removeable from a central lumen of the catheter. The system can be configured to have a second configuration that is determined based on a vasculature image from a patient. The second configuration can be determined based on an average vasculature image from a plurality of patients, such as would be used to create a library of devices. In some instances, the second configuration is determined based on an average vasculature image from a plurality of patients. Furthermore, the second configuration can be achieved through computer modeling of one or more vasculature images. The system can further comprising an aneurysm occlusion device for delivery by the catheter to the cerebral aneurysm. The catheter can have a complex curvature, e.g., a curvature that has multiple planes or three or more curves in a single plane. Additionally, the catheter can be patient-specific. Typically, the second configuration is multi-planar, such that the length between any two curves may or may not occur in the same plane as an adjacent length between an adjacent pair of curves. The catheter shape typically is determined from a plurality of images of a patient's vasculature.

Another aspect is directed to a system for accessing a cerebral aneurysm comprising: a catheter having a distal end and a proximal end and further comprising a first configuration and a second configuration wherein the first configuration is adapted and configured to be delivered through a vasculature and the second configuration is adapted and configured to assume a shape in more than one plane; and a guidewire insertable through and removeable from a central lumen of the catheter. The system can be configured to have a second configuration that is determined based on a vasculature image from a patient. The second configuration can be determined based on an average vasculature image from a plurality of patients, such as would be used to create a library of devices. In some instances, the second configuration is determined based on an average vasculature image from a plurality of patients. Furthermore, the second configuration can be achieved through computer modeling of one or more vasculature images. The system can further comprising an aneurysm occlusion device for delivery by the catheter to the cerebral aneurysm. The catheter can have a complex curvature. Additionally, the catheter can be patient-specific. Typically, the second configuration is multi-planar, such that the length between any two curves may or may not occur in the same plane as an adjacent length between an adjacent pair of curves. The catheter shape typically is determined from a plurality of images of a patient's vasculature.

Kits are also provided for to facilitate treating a blood vessel aneurysm. The kits comprise: an aneurysm treatment device adapted to be delivered to a blood vessel aneurysm; and a catheter having a distal end and a proximal end and further comprising a first configuration and a second configuration wherein the first configuration is adapted and configured to be delivered through a vasculature and the second configuration is adapted and configured to assume a shape in more than one plane. Additional components of the kit can include one or more of stents, occlusive devices or materials, a storage wire for retaining a storage shape, and sterile packaging.

Another kit provides for treating a blood vessel aneurysm and comprises: an aneurysm treatment device adapted to be delivered to a blood vessel aneurysm; and a catheter having a distal end and a proximal end and further comprising a first configuration and a second configuration wherein the first configuration is adapted and configured to be delivered through a vasculature and the second configuration is adapted and configured to assume a vasculature conformable shape. Additional components of the kit can include one or more of stents, occlusive devices or materials, a storage wire for retaining a storage shape, and sterile packaging.

Another aspect is directed to a catheter comprising: a lumen extending therethrough, a distal end and a proximal end wherein the distal end is adapted and configured to be positioned within a lumen of an aneurysm and to deliver aneurysm occlusion devices therein, wherein the catheter further comprises a first configuration and a second configuration wherein the first configuration is adapted and configured to be delivered through a vasculature and the second configuration is adapted and configured to assume a shape in more than one plane.

Methods are also included. One method includes the method of treating an aneurysm comprising: advancing a catheter having a distal end and a proximal end and further comprising a first configuration and a second configuration wherein the first configuration is adapted and configured to be delivered through a vasculature and the second configuration is adapted and configured to assume a vasculature conformable shape, in its first configuration to a target location; positioning a distal tip of the catheter within the lumen of an aneurysm; removing a guidewire from within the lumen of the catheter; wherein the catheter assumes the vasculature conformable shape after removal of the guidewire. One or more occluding devices can be delivered to the aneurysm. During the process of delivering the occluding devices, the catheter remains securely positioned within the vasculature such that the distal tip is not displaced from within the lumen of the aneurysm during delivery of the occluding devices or material. The method allows for a fill percentage greater than 25%. Furthermore, the method reduces the recurrence of aneurysms.

Another method of treating an aneurysm comprises: advancing a catheter having a distal end and a proximal end and further comprising a first configuration and a second configuration wherein the first configuration is adapted and configured to be delivered through a vasculature and the second configuration is adapted and configured to assume a shape in more than one plane, in its first configuration to a target location; positioning a distal tip of the catheter within the lumen of an aneurysm; removing a guidewire from within the lumen of the catheter; wherein the catheter assumes the vasculature conformable shape after removal of the guidewire. One or more occluding devices can be delivered to the aneurysm. During the process of delivering the occluding devices, the catheter remains securely positioned within the vasculature such that the distal tip is not displaced from within the lumen of the aneurysm during delivery of the occluding devices or material. The method allows for a fill percentage greater than 25%. Furthermore, the method reduces the recurrence of aneurysms.

Additionally, a method of making a catheter is provided for. The method of making the catheter comprises: obtaining an image of a vasculature of a patient; identifying the three-dimensional geometry of the vasculature; manufacturing a catheter having a distal end and a proximal end and further comprising a first configuration and a second configuration wherein the first configuration is adapted and configured to be delivered through a vasculature and the second configuration is adapted and configured to assume a vasculature conformable shape. The method can use a plurality of vasculature images from a plurality of patients to derive a variety of shapes to create a library of devices. Alternatively, the method can produce a patient specific catheter.

In another aspect, a method of making a catheter is provided for that comprises: obtaining an image of a vasculature of a patient; identifying the three-dimensional geometry of the vasculature; manufacturing a catheter having a distal end and a proximal end and further comprising a first configuration and a second configuration wherein the first configuration is adapted and configured to be delivered through a vasculature and the second configuration is adapted and configured to assume a shape in more than one plane. The method can be achieved using a plurality of vasculature images from a plurality of patients to achieve a library of shapes based on average sizes, curves and shapes. Alternatively, a patient specific catheter can be produced.

A mandrel is also provided for that comprises: a distal end and a proximal end and a configuration configured to impart a vasculature conformable shape to catheter.

### INCORPORATION BY REFERENCE

All publications and patent applications mentioned in this specification are herein incorporated by reference in their entirety to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-D depict a blood vessel in a mammal, such as a human, having an aneurysm therein; the aneurysm of FIG. 1A has a wide neck opening into the lumen of the blood vessel, while the aneurysm of FIG. 1B has a narrow neck opening into the lumen of the blood vessel; FIG. 1C illustrates a close-up section of blood vessel showing detail of curvature of the anatomy; FIG. 1D illustrates a view of blood vessels illustrating the large number of curves through which a catheter would navigate;

FIG. 2A depicts and overall view of a catheter device for accessing an aneurysm wherein the catheter can be adapted to have a first configuration and a second configuration; FIGS. 2B-C illustrate cross-sectional views of the catheter of FIG. 2A;

FIGS. 3A-H illustrate a variety of cross-sections of the catheter of FIG. 2A which could be employed;

FIGS. 4A-C illustrate a catheter tip entering an aneurysm (FIGS. 4A-B) and then delivering a coil (FIG. 4C) and then being removed (FIG. 4D);

FIGS. 5A-C illustrate vascular sections undergoing measurement;

FIGS. 6A-M illustrate a variety of distal ends of catheters;

FIGS. 7A-B illustrate vasculature with coils delivered into an aneurysm; and

FIGS. 8A-B are flow charts illustrating methods of the invention.

### DETAILED DESCRIPTION OF THE INVENTION

### I. Anatomy

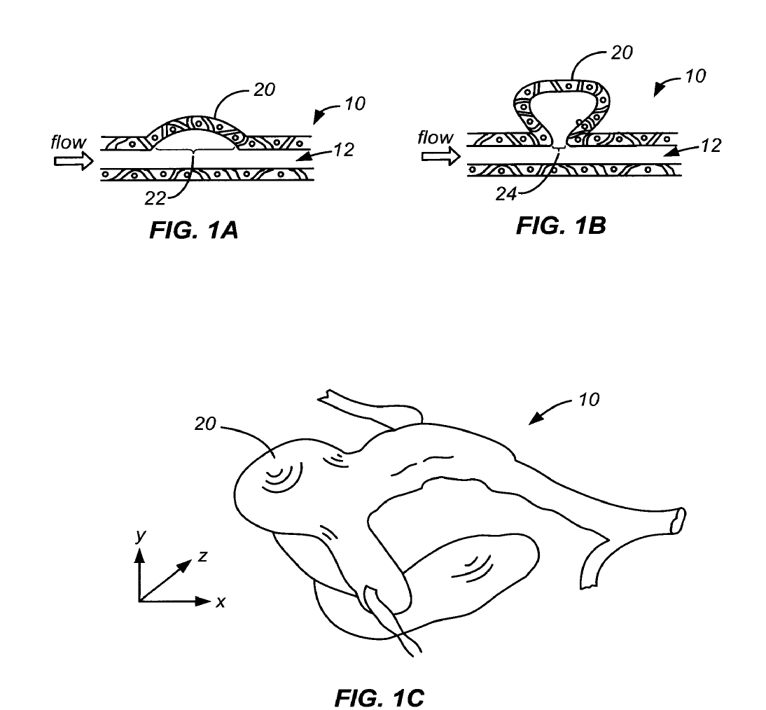

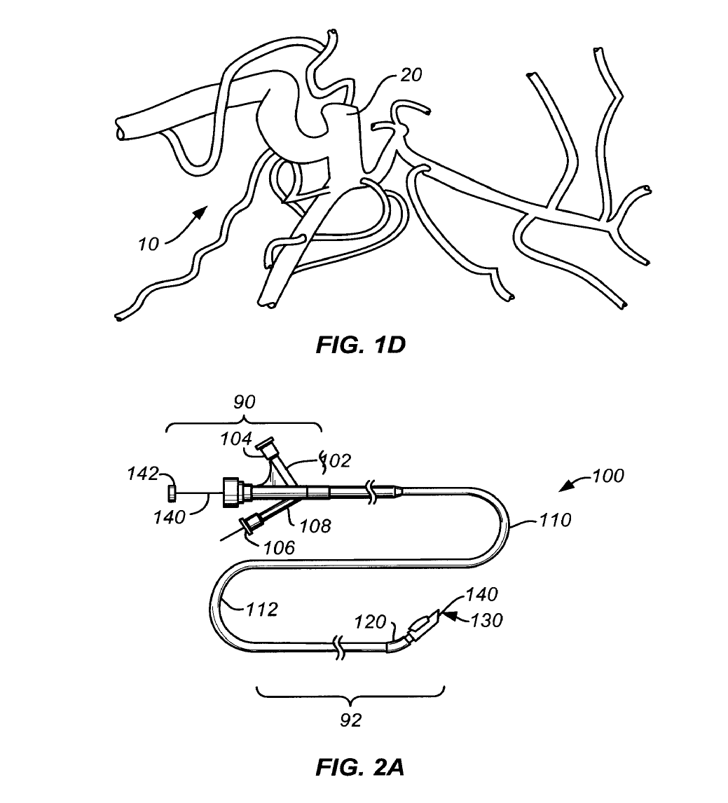

FIGS. 1A-B depict a blood vessel 10 of a mammal defining a lumen 12 having an aneurysm 20 therein. Mammals include humans, horses, dogs, and cats, to name a few. The aneurysm 20 of FIG. 1A has a wide neck opening 22 into the lumen 12 of the blood vessel 10. In contrast, the aneurysm 10 of FIG. 1B has a narrow neck 24 opening into the lumen 12 of the blood vessel 10. FIGS. 1C-D illustrates a close-up section and a remote view of a portion of tortuous vasculature 30. As will be appreciated by reviewing FIGS. 1C-D, the vasculature is full of twists, turns and bends, such that it has an overall twisting, winding or crooked appearance. As a result, it will be appreciated that access through this vasculature is not straightforward and requires intricate and circuitous steering.

### II. Catheter Devices

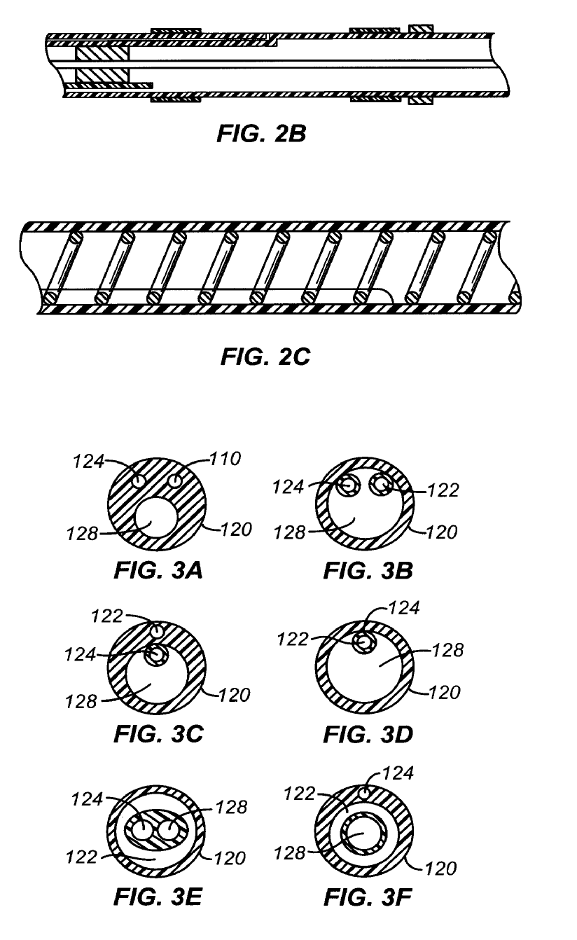

FIG. 2A-D shows a catheter assembly 100 made according to a variation suitable for use in the practice of this invention. As will be appreciated by those skilled in the art, the concepts disclosed herein are not limited to a particular catheter assembly and the catheter assembly is provided for illustration. The catheter assembly 100 includes a catheter shaft 110. The shaft can be comprised of a flexible, thin walled body or tube 112 having an inner lumen which extends between a proximal end 90 and a distal end 92. The proximal end is the end situated near the origin or near the user. The distal end is the end is situated away from the origin, or user, and is typically the end of a catheter positioned within the body. The tube 112 is preferably a generally non-distensible polymer having the appropriate mechanical properties for this application, and preferably polyethylene (e.g., high-density polyethylene (HDPE), low density polyethylene (LDPE), linear low density polyethylene (LLDPE), medium density polyethylene (MDPE), etc.), polyesters (such as nylon), polypropylene, polyimide, polyvinyl chloride, ethylvinylacetate, polyethylene terephthalate, polyurethane (e.g. Texin® thermoplastic polyurethan made by Bayer Corporation), PEBAX® thermoplastic polymer, fluoropolymers, mixtures of the aforementioned polymers, and their block or random co-polymers. One or more components can be formed of shape memory polymers, or other shape memory materials. Shape memory polymers are typically composed of two components with different thermal characteristics, oligo(ε-caprolactone)diol and crystallisable oligo(ρ-dioxanone)diol. The biodegradable multiblockcopolymer features two blockbuilding segments, a hard segment and a ‘switching’ segment, which are linked together in linear chains. The higher-temperature shape is the plastic's ‘permanent’ form, which it assumes after heating. Other shape memory materials, such as nickel titanium alloy (nitinol), or other suitable shape memory materials, can be used as well, including electrically activated shape memory materials.

The catheter can be braided or non-braided. Heating and cooling of the polymer enables the catheter to retain its shape after processing even if the catheter is not formed from a shape memory material. Typically the shape of the catheter is controlled by the shape of the mandrel, as discussed further below.

As with other catheter designs currently available, this catheter assembly has the ability to: achieve access through the vasculature to the brain (or other vascular site) often, but not necessarily, using a guide wire; can optionally include the inflation of an inflatable member or balloon to close or to restrict an artery or the mouth of an aneurysm prior to or during placement of a vaso-occlusive device, thereby requiring a fluid pathway for inflation of the inflatable member; flexion of a flexible neck in the region of the distal end of the catheter by a wire extending proximally through the catheter; and introduction of a vaso-occlusive device or material for eventual placement in the vasculature, thereby requiring a pathway or storage region for the vaso-occlusive device. These functions may be achieved by features found at the proximal and distal regions of the catheter.

The proximal catheter end 90 may be provided with a fitting 102 (e.g., a “LuerLok”) through which fluid may be supplied to the catheter's inflation lumen through a side port 104. The proximal end 90 of the catheter 100 is provided with a second port 106 and a fitting 108 through which a push/pull wire may be used to manipulate the flexible neck region 120 in the distal catheter tip 130. The proximal end fitting 102 includes an axially extending port 114 which communicates with the catheter's delivery/guide wire lumen. The optional guide wire 140 may have any suitable construction or configuration for guiding the flexible catheter shaft 110 to its target location within the body. The proximal end 90 of the guidewire 140 may be equipped with a handle or control mechanism 142 adapted to apply torque to the guidewire 140 during catheter operation. The guidewire 140 may have a variable stiffness or stepped diameter along its length to aid in steerability of the guidewire. Typically increased steerability is achieved by, for example, a configuration that uses a larger-diameter, stiffer proximal region and one or more smaller-diameter, more flexible distal regions, as will be appreciated by those skilled in the art. Other configurations can be employed without departing from the scope of the invention.

The distal portion 92 of the catheter 100 may include an articulating neck region 120, and an opening or aperture 116 for delivery of the vaso-occlusive device or material. This opening 116 may also be used for delivery of drugs and the vaso-occlusive device to the selected vascular site.

The inflatable section can be formed from a thin sleeve of polymeric material and attached at its opposite sleeve ends to a relatively more rigid tube section. Flexion of the articulating neck region 120 is achieved through remote manipulation of the push/pull wire 122 by the user.

FIG. 2B depicts a cross-section of the catheter 100 shown in FIG. 2A. A thinned region of catheter wall 212 is flanked proximally and distally by regions of greater wall cross-sectional area 214, 216. The section 216 of the catheter wall 212 acts as an articulating, flexible member when the distal end of the catheter is manipulated using the push/pull wire 122. The articulating neck allows the catheter tip to be steered with up to 360° of mobility. The variations in wall cross sectional area is typically created during an extrusion process when the device is manufactured. FIG. 2C depicts the articulating, or hinging, region 120 which utilizes a coil 222 of varying pitch imbedded in the catheter wall. Because the variation in pitch of the coil 222 produces regions of varying flexibility, the lower pitch region 224 is more flexible than the region of higher pitch 226. The higher pitch region 226 is stiffer during manipulation by the user of the push/pull wire 122.

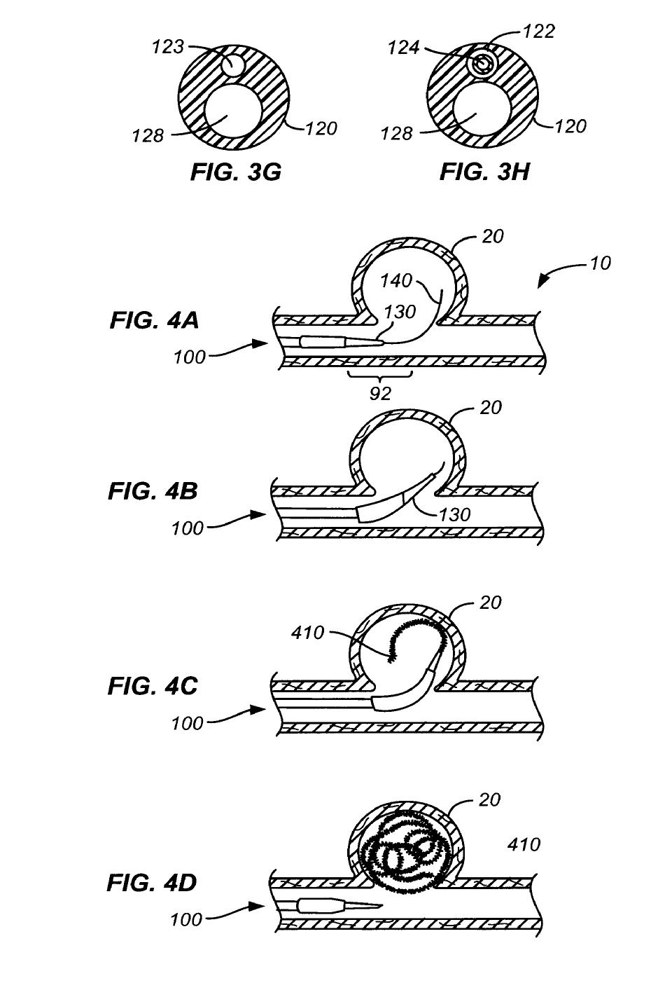

Some of the various configurations of the catheter's lumina (inflation, push/pull, and delivery) are displayed in FIGS. 3A-H. In FIG. 3A, the inflation lumen 322 and push/pull wire lumen 324 are formed interior to the catheter wall 320, while the interior catheter wall forms the guide wire lumen 328. In FIG. 3B, the catheter wall 320 forms the guide wire lumen 328 which contains the inflation lumen 322 and push/pull wire lumen 324. The inflation lumen 322 is formed interior to the catheter wall 320 of FIG. 3C, while the push/pull wire lumen 324 lies within the larger coil lumen 328 (which is formed by the catheter wall 120). FIG. 3D depicts a variation of FIG. 3C in which the push/pull wire lumen 324 lies interior to the catheter wall 328 while the inflation lumen 322 lies within the larger coil lumen 328. In FIG. 3E, the interior catheter wall 320 forms the inflation lumen 322, and the push/pull wire lumen 324 and the guide wire lumen 328 are found within the inflation lumen 322. The inflation lumen 322 surrounds the guide wire lumen 328 and lies within the region formed interior catheter wall 320 in FIG. 3F, while the push/pull wire lumen 324 lies within the catheter wall 320. In FIG. 3G, one shared lumen 323 serves as the push/pull and inflation lumen; the shared push/pull and inflation lumen 323 along with the guide wire lumen 328 lie within the catheter wall 320. Another alternate variation of the lumina positioning, shown in FIG. 3H, has the push/pull wire lumen 324 lying interior to the inflation lumen 322 which is contained within the catheter wall 320, while a separate lumina for the guide wire 328 also is contained within the catheter wall.

A storage wire may also be provided which is maintained in a lumen of the catheter during delivery and storage in order to maintain the catheter in substantially a single plane, similar to the depiction shown in FIG. 2A. Removal of the storage wire allows the catheter to return to its pre-determined multi-planar shape, as described below.

The tube constructions, hinge region construction, and other tubing forming the various lumina discussed herein may be created through extrusion, sequential production (in which the parts are manufactured separately and later assembled together), or some other method.

### III. Manufacture

A. Shaping

Shaping can be achieved by a variety of mechanism apparent to those skilled in the art. In one method, three dimensional rotational catheter angiography are used to develop a three-dimensional model of the vasculature. The three dimensional model is then used as a basis to create a three-dimensional design for the catheter wherein the catheter has two or more curves in two or more planes at its distal end. The two or more curves can correspond to vascular curves adjacent to the aneurysm, or can be major curves adjacent the aneurysm (e.g., not necessarily curves appearing in sequence). Typically a suitable imaging technique for determining the shape of the device enables the user to make a three-dimensional volumetric evaluation of the blood vessels.

The catheter itself can then be shaped by, for example, inserting a mandrel into the catheter tip and then shaping the catheter to a desired curvature. The mandrel itself can also be formed from suitable shape memory materials. The curves of the mandrel can then be exaggerated to compensate for any loss of shape that might occur, for example, when the device is deployed. Once the desired curvature is achieved, the catheter is heated using steam or some other suitable heating mechanism. The process of heating results in the catheter retaining that shape. Thereafter the mandrel is removed and the shaped catheter is ready for deployment or packaging.

Alternatively, one or more images may be taken of a patient on whom the aneurysm procedure is to be performed. In obtaining an image of the vasculature in a mammal, a number of internal imaging techniques known in the art are useful for electronically generating a vascular image. These include magnetic resonance imaging (MRI), computed tomography scanning (CT, also known as computerized axial tomography or CAT, as well as CT angiogram or CTA), magnetic resonance angiography (MRA), 3-dimensional rotational angiogram (3DRA), angiogram, and ultrasound imaging techniques. Other techniques may be apparent to one of skill in the art. As will be appreciated by those skilled in the art, different imaging techniques may require different levels of manipulation to achieve the methods descried here. A variety of imaging techniques can be used to achieve the shaping, including:

CT/CTA

A computed tomography (CT) or computer axial tomography (CAT) scan uses X-rays to make detailed pictures of structures inside of the body. The CT uses multiple images, each in a single plane, or slice, of to obtain a tomogram. Following injection of contrast intravenously the vasculature will they come denser. The images can then be compiled to generate a three-dimensional representation of vasculature architecture for a patient. Alternatively this can be performed in two steps one prior to injection of the contrast and one after and subtracted from each other. The Computer is then able to make three-dimensional images of the blood vessels which can be rotated in any direction.

MRA

MRA enables imaging of blood vessels. Typically pictures of arteries are generated in order to evaluate the arteries for stenosis (abnormal narrowing) or aneurysms (vessel wall dilations). MRA is most often used in evaluating the arteries of the neck and brain. A variety of techniques can be used to generate pictures. For example, paramagnetic contrast agents such as gadolinium can be used. Additionally, flow related enhancement techniques can be used where most of a signal on an image is due to blood which has recently moved into the plane.

Angiogram/3-D Rotational Angiogram

The angiogram is an x-ray picture taken to visualize the inner opening of a blood filled structure, such as an artery or vein. Angiograms are achieved with the use of a catheter. The images may be taken as still images, e.g. x-ray, or displayed on fluoroscopic film. The images can also be obtained volumetrically prior and after contrast injection once subtracted from each other and or alternatively only after contrast injection and manipulated based on the density. With this data to three-dimensional images of the blood vessels are made and can be rotated in any directions

MRI

MRI can be used to understand the direction of the blood vessels. As techniques change, it may also become suitable for making a volumetric evaluation of blood vessels. One advantage of MRI is good contrast between various types of tissue, including cartilage, bone, joint fluid, ligaments, muscle, blood and moving material such as blood and blood vessels which facilitates a delineation and segmentation of data sets. Another advantage is the coverage of the entire region of interest in a single scan within acceptable acquisition times. See, MRI Basic Principles and Applications, Second Edition, Mark A. Brown and Richard C. Semelka, Wiley-Liss, Inc. (1999).

MRI employs pulse sequences that allow for better contrast of different parts of the area being imaged. Different pulse sequences are better fitted for visualization of different anatomic areas, for example, hyaline cartilage or joint fluid. More than one pulse sequence can be employed at the same time. A brief discussion of different types of pulse sequences is provided below.

High Resolution 3D MRI Pulse Sequences

Routine MRI pulse sequences available for imaging tissue include conventional T1 and T2-weighted spin-echo imaging, gradient recalled echo (GRE) imaging, magnetization transfer contrast (MTC) imaging, fast spin-echo (FSE) imaging, contrast enhanced imaging, rapid acquisition relaxation enhancement, (RARE) imaging, gradient echo acquisition in the steady state, (GRASS), and driven equilibrium Fourier transform (DEFT) imaging. As these imaging techniques are well known to one of skill in the art, e.g. someone having an advanced degree in imaging technology, each is discussed only generally hereinafter. While each technique is useful for obtaining a cartilage degeneration pattern, some are better than others.

Conventional T1 and T2-Weighted Spin-Echo Imaging

Conventional T1 and T2-weighted MRI depicts vasculature, and can demonstrate defects and gross morphologic changes.

Gradient-Recalled Echo Imaging

Gradient-recalled echo imaging has 3D capability and ability to provide high resolution images with relatively short scan times.

Fast Spin-Echo Imaging

Fast spin-echo imaging may be another useful pulse sequence to evaluate vasculature. Incidental magnetization transfer contrast contributes to the signal characteristics of vasculature on fast spin-echo images and can enhance the contrast between the vasculature and surrounding tissue.

B. Sizing

The microcatheter can be formed or selected so that it will achieve a near anatomic fit with all or part of the vasculature through which it travels to correspond to the twists and turns of the surrounding or adjacent vessel walls. The shape of the catheter can be based on the analysis of an electronic image (e.g. MRI, CT, angiography, digital tomosynthesis, optical coherence tomography or the like) for a particular patient and therefore be patient specific, or can be based on an analysis of a plurality of patients, or a plurality of patients within a specific criteria, to achieve an average three-dimensional shape that is directed to the identified vasculature architecture. A near anatomic shape tracking can be achieved using a method that provides a virtual reconstruction of the shape of the vasculature in one or more electronic images.

In one embodiment of the invention, a vascular architecture can be reconstructed by interpolating the images. Alternatively, the vasculature can be reconstructed using morphological image processing technique. In a first step, the vascular image can be extracted from the electronic image using manual, semi-automated and/or automated segmentation techniques (e.g., manual tracing, region growing, live wire, model-based segmentation), resulting in a binary image. Vascular sizing can be performed in 2-D or 3-D with an appropriately selected structuring element.

As described above, the catheter can be formed or selected from a library or database of systems of various sizes and curvatures so that it will achieve a near anatomic fit or match with the surrounding or adjacent vasculature. These systems can be pre-made or made to order for an individual patient. In order to control the fit or match of the catheter system with the surrounding or adjacent vasculature, a software program can be used that projects the vascular system over the vascular system where it will be used. Suitable software is commercially available and/or readily modified or designed by a skilled programmer.

In yet another embodiment, the catheter system can be projected over the vascular system where the catheter will use used using one or more 3-D images. The vasculature and other anatomic structures that might be of interest are extracted from a 3-D electronic image such as an MRA, 3-D rotational angiogram or a CTA using manual, semi-automated and/or automated segmentation techniques. A 3-D representation of the vasculature and other anatomic structures as well as the catheter system is generated, for example using an angiogram machine, such as the Philips Allura Xper FD20/10 available from Philips Medical Systems. For a description of various parametric surface representations see, for example Foley, J. D. et al., Computer Graphics: Principles and Practice in C; Addison-Wesley, 2.sup.nd edition, 1995).

The 3-D representations of the vasculature and other anatomic structures and the catheter system can be merged into a common coordinate system. The catheter system can then be placed at the desired treatment site. The representations of the anatomic structures and the catheter system are rendered into a 3-D image, for example application programming interfaces (APIs) OpenGL®. (standard library of advanced 3-D graphics functions developed by SGI, Inc.; available as part of the drivers for PC-based video cards, for example from www.nvidia.com for NVIDIA video cards or www.3dlabs.com for 3Dlabs products, or as part of the system software for Unix workstations) or DirectX® (multimedia API for Microsoft Windows® based PC systems; available from www.microsoft.com). The 3-D image can be rendered showing the vasculature or other anatomic objects, and the catheter system from varying angles, e.g. by rotating or moving them interactively or non-interactively, in real-time or non-real-time.

The software can be designed so that the catheter system with the best fit relative to the vasculature is automatically selected, for example using some of the techniques described above. Alternatively, the operator can select a catheter system, and project it or drag it onto the target vascular site using suitable computer tools and techniques. The operator can move and rotate the catheter systems in three dimensions relative to the target vascular site and can perform a visual inspection of the fit between the catheter system and the target vascular site. The visual inspection can be computer assisted. The procedure can be repeated until a satisfactory fit has been achieved. The procedure can be performed manually by the operator; or it can be computer-assisted in whole or part. For example, the software can select a first trial catheter that the operator can test. The operator can evaluate the fit. The software can be designed and used to highlight areas of poor alignment between the catheter and the surrounding target vasculature. Based on this information, the software or the operator can then select another catheter and test its alignment. One of skill in the art will readily be able to select, modify and/or create suitable computer programs for the purposes described herein.

In another embodiment, the target vascular site can be visualized using one or more cross-sectional 2-D images. Typically, a series of 2-D cross-sectional images will be used. The 2-D images can be generated with imaging tests such as angiogram, CTA, MRA, CT, MRI, digital tomosynthesis, ultrasound, or optical coherence tomography using methods and tools known to those of skill in the art. The catheter system can then be superimposed onto one or more of these 2-D images. The 2-D cross-sectional images can be reconstructed in other planes, e.g. from sagittal to coronal, etc. Isotropic data sets (e.g., data sets where the slice thickness is the same or nearly the same as the in-plane resolution) or near isotropic data sets can also be used. Multiple planes can be displayed simultaneously, for example using a split screen display. The operator can also scroll through the 2-D images in any desired orientation in real time or near real time; the operator can rotate the imaged tissue volume while doing this. The catheter system can be displayed in cross-section utilizing different display planes, e.g. sagittal, coronal or axial, typically matching those of the 2-D images demonstrating the cartilage, subchondral bone, menisci or other tissue. Alternatively, a three-dimensional display can be used for the catheter system. The 2-D electronic image and the 2-D or 3-D representation of the catheter system can be merged into a common coordinate system. The catheter system can then be placed at the desired target vascular site. The series of 2-D cross-sections of the anatomic structures, the target vascular site and the catheter system can be displayed interactively (e.g. the operator can scroll through a series of slices) or non-interactively (e.g. as an animation that moves through the series of slices), in real-time or non-real-time.

### IV. Surgical Techniques

Prior to performing the procedure on a patient, the surgeon can preoperatively analyze the vasculature of the patient, for example, using CTA and MRA images. Using standard surgical techniques, the patient is anesthetized and an incision is made in order to provide access to the vasculature. Once an appropriate sized incision has been made (typically at the common femoral artery), a guiding catheter is advanced into, for example, the cervical carotid artery in the usual fashion (with or without a wire or even exchanged over a wire after a diagnostic catheter first made access into the carotid artery). A microcatheter over a micro wire is then advanced through the guiding catheter. Alternatively the guiding catheter can be advanced further if it is flexible enough and similar techniques described above can be used to shape the guiding catheter is well to provide more stability. The microcatheter is advanced over the wire to get it close to the aneurysm and also provide stiffness. The wire is then pulled back and they've microcatheter is advanced into the aneurysm with or without wire assist.

FIGS. 4A-D illustrate a remotely flexible distal tip treating an aneurysm by placement of a vaso-occlusive device, such as coils or other material in the aneurysm. The vaso-occlusive devices typically are designed to occlude a space within the body, e.g. within the aneurysm space. For example, vaso-occlusive devices include metallic devices such as platinum or devices having a metallic core or core member, and two polymeric members of differing thrombogenicity. The core member typically comprises a metallic helically-wound coil, the first polymeric member and second polymeric member could be fibrous materials, e.g., materials woven into a braid. The devices are then placed at the desired site within a mammal to facilitate the formation of an occlusion. Typically vaso-occlusive devices promote the formation of scar tissue, healing tissue, or neocapillaries in vascular occlusions made by the device. Vaso-occlusive devices can also include any foreign body, such as needles, that are inserted into an aneurysms to induce thrombosis. The vaso-occlusive devices can also be a suitable liquid which then hardens or thrombuses. A suitable liquid vaso-occlusive device includes glue. The various designs of the catheter disclosed herein facilitate maintaining the distal tip of the catheter within the aneurysm, which allows the physician to achieve a fill rate of the aneurysm greater than 25%. Additionally, the designs allow the tip to be positioned within the aneurysm and to achieve distal tip movement in a spherical motion across a plurality of planes, instead of less than 180° movement in a single plane, to facilitate accurate placement of the tip within the aneurysm. Furthermore, the shaping of the catheter results in the distal tip of the catheter automatically migrating toward a position within the aneurysm during deployment as a result of the catheter assuming the shape determined during process and retained as a shape memory by the device.

FIG. 4A depicts a catheter 100 that has its distal end positioned outside the mouth of an aneurysm 20 to deliver a vaso-occlusive coil. The device is positioned using a guidewire 140. The catheter's distal end 92 is introduced into the aneurysm neck 22, as shown in FIG. 4B. Flexion or steerability of the catheter's distal tip using the push/pull wire allows for greater maneuverability when accessing the aneurysm neck and aneurysm sac. As a result of the design, the distal tip of the catheter can be oriented in the plane of the aneurism opening. Thus allowing the tip of the catheter to then be placed within the aneurysm. The push/pull wire system allows the distal end to be positioned as desired during the procedure, instead of before the procedure begins. These type of catheters currently only have the ability to turn their most distal end and 180° within a single plane by putting secondary, tertiary or three-dimensional curves proximal to the distal curve. This configuration orients the rotation of the distal tip from side-to-side and will not help accessing an aneurysm which is inferior in location. If the catheter had other curvatures more proximally the rotation of the tip can be oriented up and down so it could access the aneurysm. A balloon can also be provided to assist in holding the catheter in place while the aneurysm is being treated although it is generally accepted that once the balloon is used this will increase the risk of the procedure (increased chance of thrombus and thromboembolic phenomenon and may need anticoagulation. Full occlusion of the aneurysm neck 22 may be desirable in some instances to ensure that the coils 410 do not escape the aneurysm space and enter into the vessel itself when the coils are discharged into the aneurysm sac. Once the coil or coils 410 have been completely discharged into the aneurysm sac the catheter's distal end is retracted from the aneurysm, as shown in FIG. 4D.

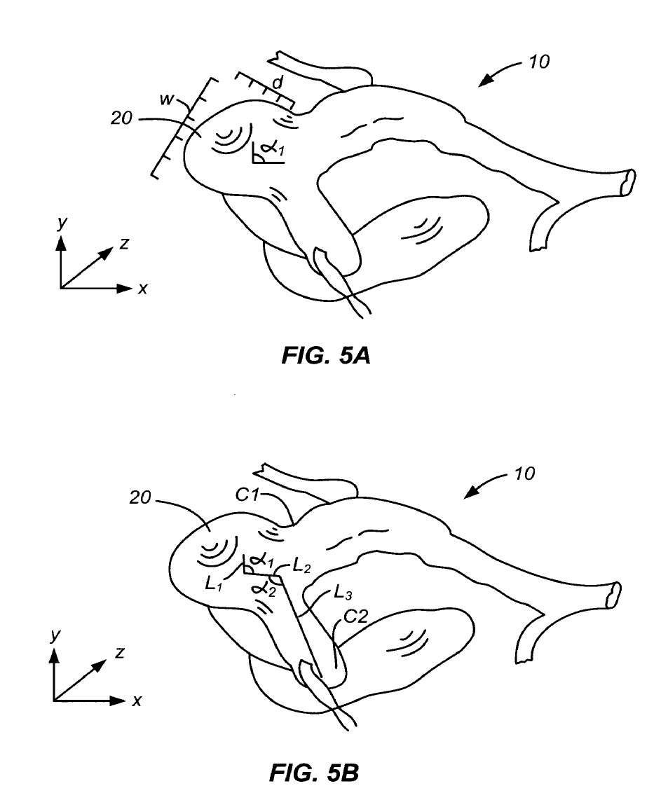

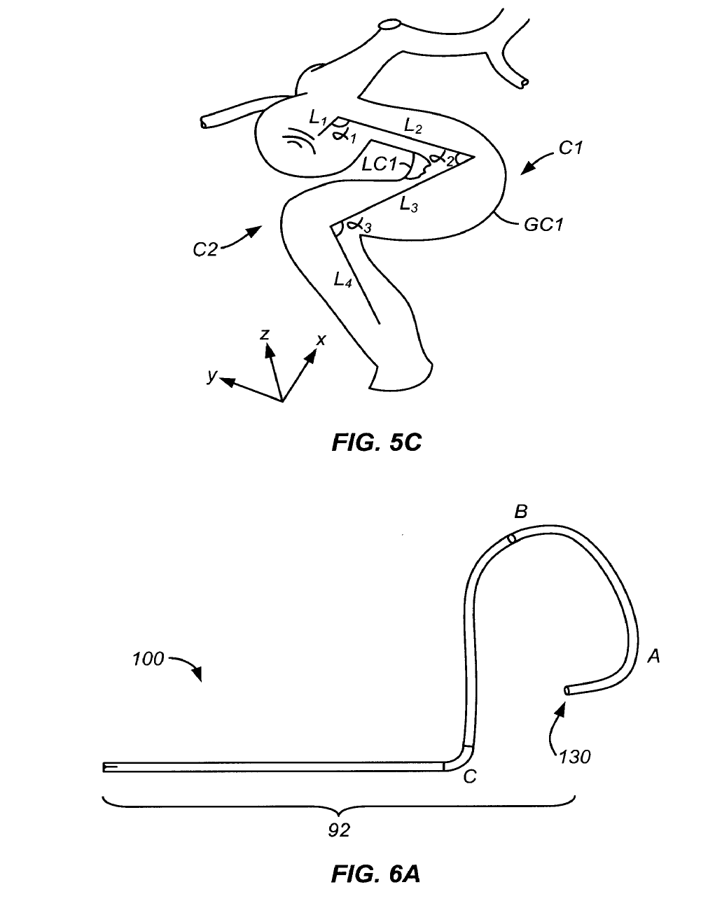

FIGS. 5A-C illustrate a variety of views of tortuous vasculature with measurements of the bends and curves of the vasculature. FIG. 5A illustrates an aneurysm 20 in vasculature 10. The view is taken, for example, at a rotation of −17 and angle of −43 from an orthogonal plane. The aneurysm has a first measurement L1 that corresponds to the distal tip 130 positioned within the aneurysm. The length L1 thus is from within the aneurysm to the artery wall outside the patent artery. A second length L2 corresponds to the distance from the aneurysm 20 to the first curve C1 of the vasculature. A third length corresponds to the distance from the first curve C1 to a second curve C2. The second curve can be the actual second curve in the vasculature, or can correspond to a second major curve. At each curve, the catheter will extend past the center of the artery toward the wall of the artery such that it will engage the greater curvature of the bend of the artery at that point. Additionally, the depth D and width W of the aneurysm can be measured to determine an optimal distal tip length prior to the first curve, thus positioning the distal tip within the aneurysm space. In this instance, the width at the neck of the aneurysm could be, for example, 6.95 mm, and at its widest section 9.22 mm, with a depth of 7.97 mm. Length L1 and L2 have an angle α1 between them. Length L2 and L3 have an angle α2 between them. Angles α1 and α2 may be similar angles and may occur within the same plane. However, in most instances, the angles are not the same and occur in separate planes. Additionally L1, L2 and L3 will have different lengths. In most situations, each of the lengths will have separate values. Fir example, the length L2 is 8.71 mm and L2 is 10.47 mm FIG. 5C, the rotation of the vasculature shown in FIG. 5A is turned to a rotation of −63° and an angle of −9°. In this depiction, four lengths are measured L1-4, with angles between each length. As will be appreciated by those skilled in the art, the shape of the actual curves will not necessarily be in a single plane as a result of the three-dimensional curvatures occurring in the vasculature. The greater curve GC of the bend for curve C1 opposes the lesser curve LC of C1. Allowing the catheter to at least partially abut the greater curve vascular wall, further enables the catheter to maintain its position during delivery of the vaso-occlusive devices.

Using information on curvature of the vasculature, a physical model of the surfaces of the vasculature can be created. This physical model can be representative of a limited section of the vasculature encompassing or adjacent to the aneurysm, or can be a model of substantially the entire vasculature. This model can also take into consideration the presence or absence of brain, muscle, bone or other tissue. The location of the aneurysm can be included in the model, for example using a 3D coordinate system or a 3D Euclidian distance. Typically, the model includes the region encompassing all or part of the aneurysm along with a section of vasculature having two or more bends prior to the aneurysm. In this way, an optical curvature of the catheter can be determined.

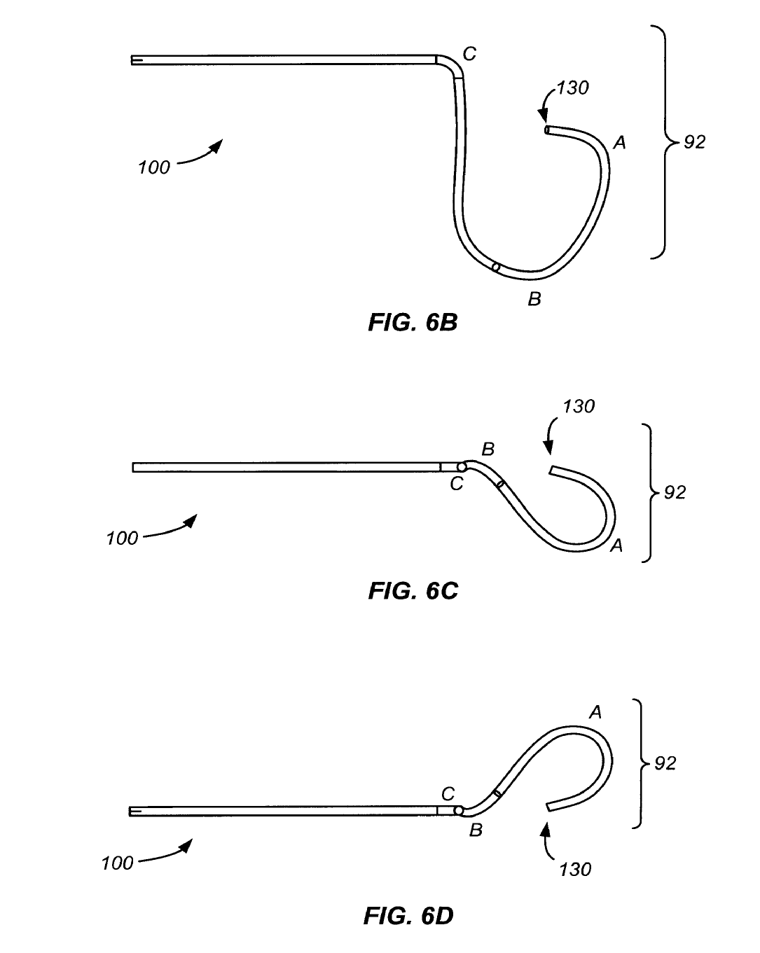

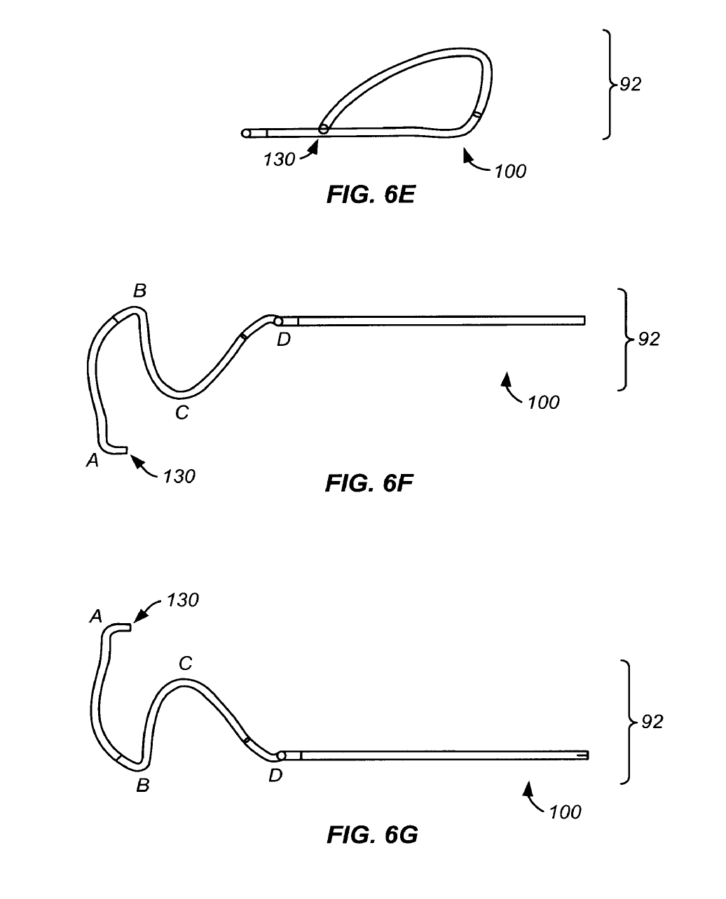

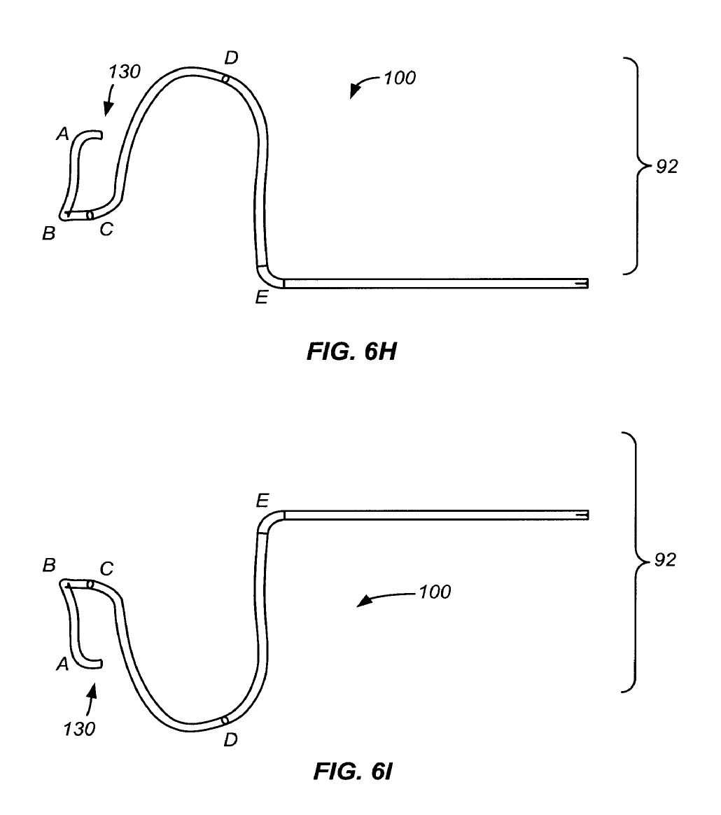

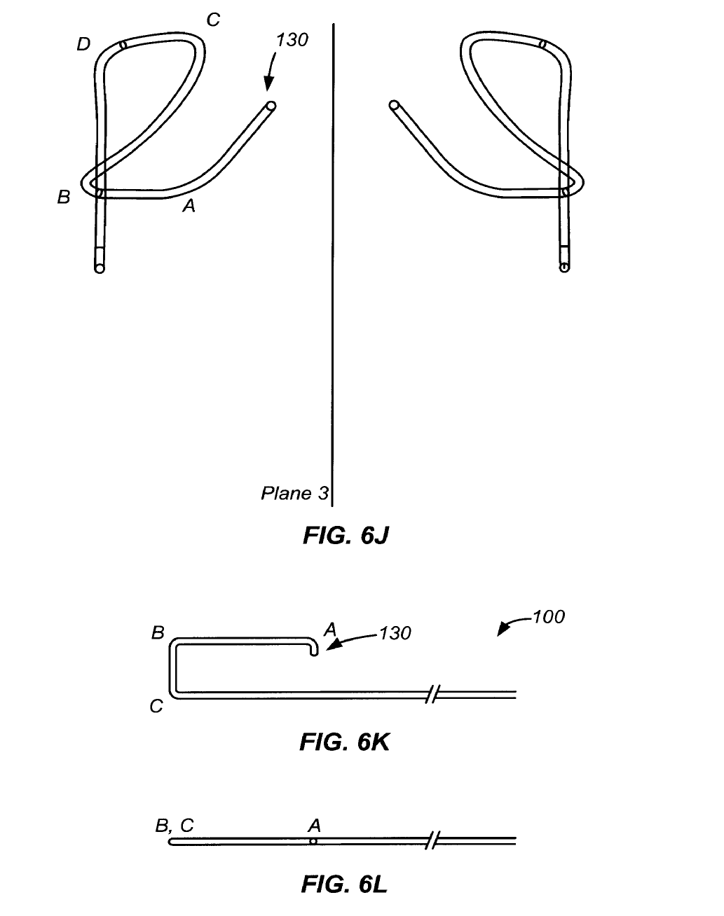

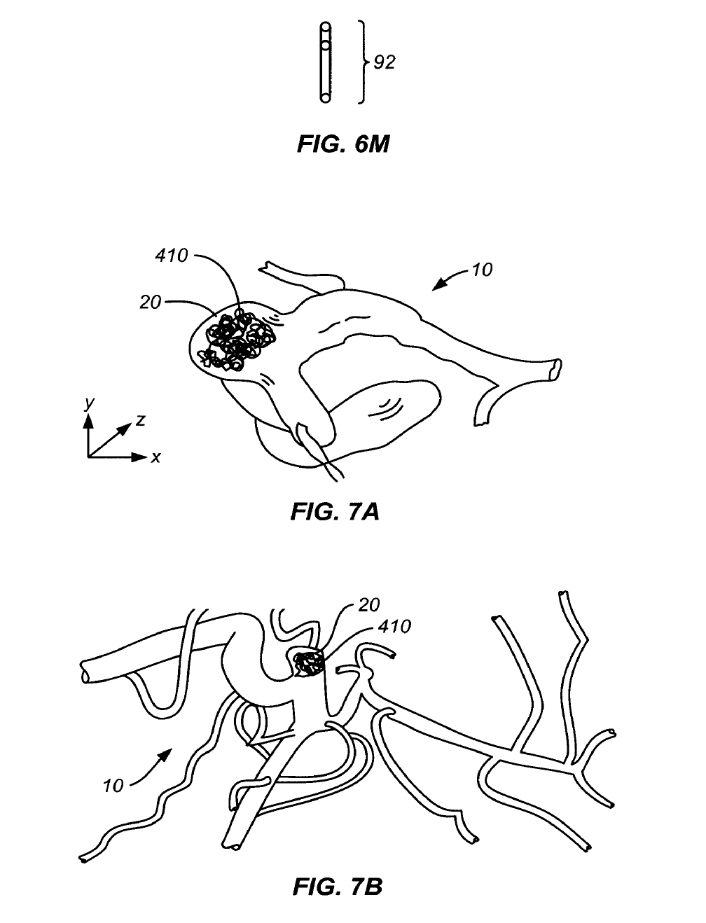

FIGS. 6A-M illustrate a variety of patient-specific distal end configurations achievable for catheters manufactured according to the methods disclosed herein. Each of the catheter configurations has complex curvatures, i.e., curvatures that occur in multiple planes or catheters which have three or more curves in a single plane. FIGS. 6A-E illustrate views of a catheter 100 adapted and configured to access a posterior communicating artery (“pcomm”) aneurysm. The pcomm is one of a pair of right-sided and left-sided blood vessels in the circle of Willis. It connects the three cerebral arteries of the same side. Anteriorly, it is one portion of the terminal trifurcation of the internal carotid artery. The anterior cerebral artery and the middle cerebral artery are the other two branches of the trifurcation. Posteriorly, the pcomm communicates with the posterior cerebral artery. FIG. 6A illustrates a right side view having curves A, B, C. FIG. 6B illustrates the same catheter from a left side view. FIGS. 6C-D illustrate a frontal view of the catheter which comes out of the plane of the page. FIG. 6E illustrates a top view of the catheter from the right side.

FIGS. 6F-J illustrate views of a catheter 100 adapted and configured to access an anterior communicating artery (“acomm”) aneurysm. The acomm is a blood vessel of the brain that connects the left and right anterior cerebral arteries. The acomm connects the two anterior cerebral arteries across the commencement of the longitudinal fissure and is a common location of aneurysms. Sometimes this vessel is wanting, the two arteries joining together to form a single trunk, which afterward divides; or it may be wholly, or partially, divided into two. Its length averages about 4 mm, but varies greatly. It gives off some of the anteromedial ganglionic vessels, but these are principally derived from the anterior cerebral artery.

FIGS. 6K-M illustrates a catheter adapted and configured to access a pcomm artery having three curves, A, B and C. This embodiment provides for a catheter design in a single plane. FIG. 6K illustrates a lateral view, FIG. 6L illustrates a superior view and FIG. 6M illustrates an anterior view.

The various curves shown in the embodiments of FIG. 6 include curves at, for example, A, B, C, and D. The length from the distal tip 130 to curve A, from curve A to curve B, curve B to curve C, and curve C to curve D can, as illustrated here, be of different lengths. From the distal tip 130 to the first curve (A), the catheter is designed to extend out of the aneurism to the opposing wall of the parent artery. The catheter is configured such that it assumes a configuration that positions the distal end of the catheter within the aneurysm and secures the catheter within the vasculature such that the catheter is adapted and configured to deliver vaso-occlusive devices without being dislodged from the aneurysm. From then on the catheter will tend to hug the greater curvatures of the artery (i.e., the outer wall of any curve). Additionally, although depicted herein as a two-dimensional shape in a single plane, in practice the curves and lengths would not necessarily be in the same plane. The custom curves and lengths allow the catheter to anchor within the vasculature to provide control the position of the distal tip relative to the vasculature and prevent the distal tip from being dislodged from within the aneurysm as the vaso-occusive devices are delivered. For at least some configurations, during delivery, once the guidewire is removed, the catheter's shape memory properties will cause it to assume the correct position within the vascular and will position the distal tip of the catheter within the lumen of the aneurysm without further manipulation.

FIGS. 7A-B illustrate the vasculature 10 with the vaso-occlusive devices, such as coils, delivered into the aneurysm 20.

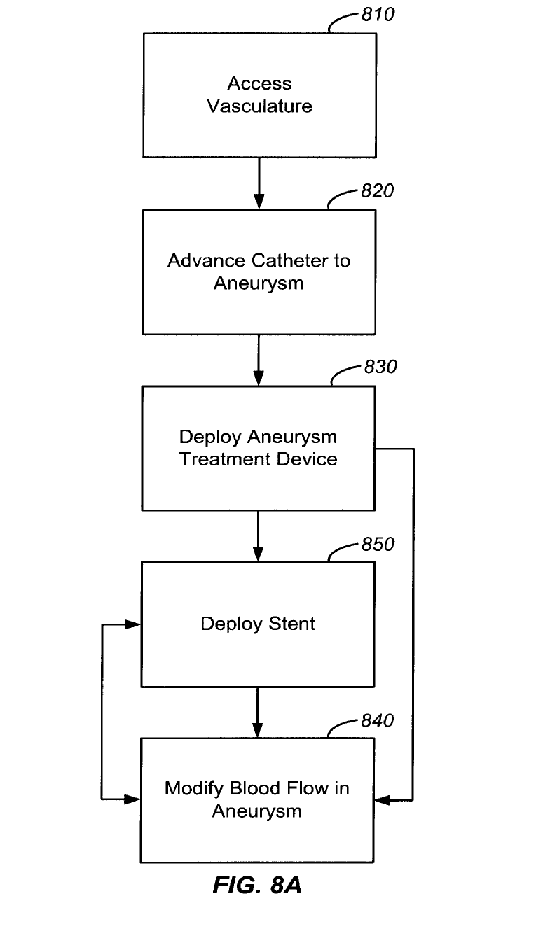

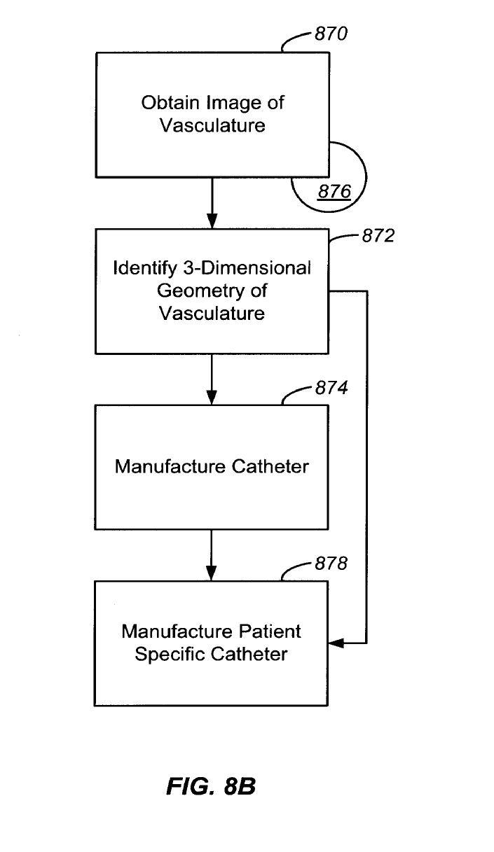

As illustrated in FIG. 8A, the invention also includes a method for treating a blood vessel aneurysm. The method includes: accessing a vasculature 810; advancing a catheter adapted to engage an aneurysm treatment device at a distal tip through the vasculature to reach the aneurysm 820; and deploying the aneurysm treatment device from the distal tip of the catheter at the aneurysm to modify blood flow at the aneurysm 830. In some embodiments of the method, a stent can be deployed 850 within the vasculature adjacent the aneurysm. The method of the invention can result in partially occluding a neck of the aneurysm and/or modifying the blood flow in an aneurysm 840. As will be appreciated by those skilled in the art, the order of the steps of the method can be varied without departing from the scope of the invention. For example, after deploying the aneurysm treatment device 830, modification of the blood flow can occur at the aneurysm 840. Alternatively, concurrently, or prior to the step of modifying the blood flow, a stent can be deployed adjacent the aneurysm 850. An additional alternative could be the step of anchoring the aneurysm treatment device 860 following the step of deploying the aneurysm treatment device 830. The method allows for a fill percentage greater than 25%. Furthermore, the method reduces the recurrence of aneurysms. The methods can reduce the recurrence of aneurysms because of the improved fill percentage.

FIG. 8B illustrates a method of making a catheter. The method of making the catheter comprises: obtaining an image of a vasculature of a patient 870; identifying the three-dimensional geometry of the vasculature 872; manufacturing a catheter 874 having a distal end and a proximal end and further comprising a first configuration, e.g. a configuration that is caused by a wire straightening a catheter into a less complex shape to that it can be navigated toward or near the aneurysm, and a second configuration wherein the first configuration is adapted and configured to be delivered through a vasculature and the second configuration is adapted and configured to assume a vasculature conformable shape. The method can use a plurality of vasculature images 876 from a plurality of patients to derive a variety of shapes to create a library of devices. Alternatively, the method can produce a patient specific catheter 878.

### V. Kits

The devices herein can be made as patient-specific devices based on an analysis of a specific patient's vasculature, or can be selected from a library of devices. Kits include a pre-formed mandrel, a catheter, such as a catheter having a pre-determined shape which includes curves in more than one plane. A storage wire is provided to keep the catheter in a flat plane during shipment and storage. The catheters are typically stored under sterile conditions, as would be appreciated by those skilled in the art. Removal of the storage wire allows the catheter to regain its three dimensional, multi-planar shape. One or more guidewires can be provided to facilitate steering the catheter to a target location. Furthermore, a plurality of vaso-occlusive devices can be provided for delivery into an aneurysm by the catheter. The catheters of the kits can be pre-shaped based on patient specific data and then shipped to a recipient. Alternatively, the catheters of the kits are selected from a library of devices.

### Example 1

Fifteen consecutive patients with known cerebral vascular pathology were examined by catheter rotational angiography. These patients then underwent treatment with endovascular coils. On the workstation, the arterial segments beginning at the aneurysm and extending proximally for the next three major curves were measured from greatest curvature to greatest curvature. The microcatheter forming wire was then bend in 3-dimensions to reflect those measurements. The resulting curve was then exaggerated to allow for the expected straightening when the microcatheter would be introduced into the bloodstream.

The forming ware was then placed in the microcatheter, and these curves were set in the tip of the microcatheter with a steam generator. The finished microcatheter had a plurality of complex curves in multiple planes.

The time needed to reconstruct arteries on current workstations takes only minutes. Measurements were achieved in less than one minute and on average in less than 5 minutes. All 15 patients had satisfactory endovascular treatment of their aneurysm. No catheter backed-out of the aneurysm and no patient suffered a complication. The time needed to perform an endovascular procedure using conventionally available microcatheters can range from about 2 to about 7 minutes. The amount of time required to perform these 15 procedures with the complex curves ranged from 1 to 4 minutes. The amount of time was reduced 40% or more, which, among other things, decreases the amount of radiation a patient is subjected to during a procedures.

Using a complex shape microcatheter not only can be easier to access an aneurysm but also will help better filling of the aneurysm. The catheter or hugs the greater curvatures of the artery therefore it is more stable in the aneurysm this allows that are packing density. For example if the back of the first curve of the aneurysm is not against the opposite wall of the artery from the aneurysm as the coils are pushed forward the catheter is pushed backward. A similar concept applies for remainder of the curvatures.

### Example 2

A group of patients with pre-determined characteristics would be chosen. The characteristics can be selected from genetic markers, sex, race, ethnicity, BMI, age, etc. Each patient is then examined by catheter rotational angiography to assess vascular anatomy. On the workstation, similar arterial segments for the patients are compared for three or more major curves. The results are analyzed and compared to identify similar structures and to put together a library of catheters having three or more curves in two or more planes. The microcatheter forming wire having shape memory properties is then bend in 3-dimensions to reflect those measurements but would allow for storage in two dimensions. The resulting curve is then exaggerated to allow for the expected straightening when the microcatheter is introduced into the bloodstream.

The forming ware was then placed in the microcatheter, and these curves would be set in the tip of the microcatheter with a steam generator. The finished microcatheter would have a plurality of complex curves in multiple planes.

A physician could then analyze the vasculature of a patient having an aneurysm and select an aneurysm access device from a library of devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A system for accessing a cerebral aneurysm comprising:
a catheter having a distal end and a proximal end and further comprising a first configuration and a second configuration wherein the first configuration is adapted and configured to be delivered through a vasculature and the second configuration is adapted and configured to assume a vasculature conformable shape; and
a guidewire insertable through and removeable from a central lumen of the catheter.

2. The system of claim 1 wherein the second configuration is determined based on a vasculature image from a patient.

3. The system of claim 1 wherein the second configuration is determined based on an average vasculature image from a plurality of patients.

4. The system of claim 1 wherein the second configuration is achieved through computer modeling of one or more vasculature images.

5. The system of claim 1 further comprising an aneurysm occlusion device for delivery by the catheter to the cerebral aneurysm.

6. The system of claim 1 wherein the catheter has a complex curvature.

7. The system of claim 1 wherein the catheter is a patient-specific catheter.

8. The system of claim 1 wherein the second configuration is multi-planar.

9. The system of claim 1 wherein the second configuration further comprises three or more curves.

10. The system of claim 1 wherein the second configuration of the catheter is determined from an analysis of a plurality of images of patient vasculature.

11. The system of claim 1 further comprising a mandrel.

12. A system for accessing a cerebral aneurysm comprising:
a catheter having a distal end and a proximal end and further comprising a first configuration and a second configuration wherein the first configuration is adapted and configured to be delivered through a vasculature and the second configuration is adapted and configured to assume a shape in more than one plane; and
a guidewire insertable through and removeable from a central lumen of the catheter.

13. The system of claim 12 wherein the second configuration is determined based on a vasculature image from a patient.

14. The system of claim 12 wherein the second configuration is determined based on an average vasculature image from a plurality of patients.

15. The system of claim 12 wherein the second configuration is achieved through computer modeling of one or more vasculature images.

16. The system of claim 12 further comprising an aneurysm occlusion device for delivery by the catheter to the cerebral aneurysm.

17. The system of claim 12 wherein the catheter has a complex curvature.

18. The system of claim 12 wherein the catheter is a patient-specific catheter.

19. The system of claim 12 wherein the second configuration is multi-planar.

20. The system of claim 12 wherein the second configuration further comprises three or more curves.

21. The system of claim 12 wherein the second configuration of the catheter is determined from an analysis of a plurality of images of patient vasculature.

22. The system of claim 12 further comprising a mandrel.

23. A kit for treating a blood vessel aneurysm comprising:
an aneurysm treatment device adapted to be delivered to a blood vessel aneurysm; and
a catheter having a distal end and a proximal end and further comprising a first configuration and a second configuration wherein the first configuration is adapted and configured to be delivered through a vasculature and the second configuration is adapted and configured to assume a shape in more than one plane.

24. The kit of claim 23 further comprising a stent.

25. The kit of claim 23 further comprising one or more aneurysm occlusion devices.

26. The kit of claim 23 further comprising a storage wire for retaining the catheter in a single plane during shipping and storage.

27. The kit of claim 23 further comprising sterile packaging.

28. The kit of claim 23 further comprising a mandrel.

29. The kit of claim 23 wherein the catheter is a patient specific catheter.

30. A kit for treating a blood vessel aneurysm comprising:
an aneurysm treatment device adapted to be delivered to a blood vessel aneurysm; and
a catheter having a distal end and a proximal end and further comprising a first configuration and a second configuration wherein the first configuration is adapted and configured to be delivered through a vasculature and the second configuration is adapted and configured to assume a vasculature conformable shape.

31. The kit of claim 30 further comprising a stent.

32. The kit of claim 30 further comprising one or more aneurysm occlusion devices.

33. The kit of claim 30 further comprising a storage wire for retaining the catheter in a single plane during shipping and storage.

34. The kit of claim 30 further comprising sterile packaging.

35. The kit of claim 30 further comprising a mandrel.

36. The kit of claim 30 wherein the catheter is a patient specific catheter.

37. The kit of claim 30 wherein the catheter is selected from a library of catheter configurations.

38. A catheter comprising:
a lumen extending therethrough, a distal end and a proximal end wherein the distal end is adapted and configured to be positioned within a lumen of an aneurysm and to deliver aneurysm occlusion devices therein, wherein the catheter further comprises a first configuration and a second configuration wherein the first configuration is adapted and configured to be delivered through a vasculature and the second configuration is adapted and configured to assume a shape in more than one plane.

39. The catheter of claim 38 wherein the second configuration is determined based on a vasculature image from a patient.

40. The catheter of claim 38 wherein the second configuration is determined based on an average vasculature image from a plurality of patients.

41. The catheter of claim 38 wherein the second configuration is achieved through computer modeling of one or more vasculature images.

42. The catheter of claim 38 further comprising an aneurysm occlusion device for delivery by the catheter to the cerebral aneurysm.

43. The catheter of claim 38 wherein the catheter has a complex curvature.

44. The catheter of claim 38 wherein the catheter is a patient-specific catheter.

45. The catheter of claim 38 wherein the second configuration is multi-planar.

46. The catheter of claim 38 wherein the second configuration of the catheter is determined from an analysis of a plurality of images of patient vasculature.

47. A method of treating an aneurysm comprising:
advancing a catheter having a distal end and a proximal end and further comprising a first configuration and a second configuration wherein the first configuration is adapted and configured to be delivered through a vasculature and the second configuration is adapted and configured to assume a vasculature conformable shape, in its first configuration to a target location;
positioning a distal tip of the catheter within the lumen of an aneurysm;
removing a guidewire from within the lumen of the catheter;
wherein the catheter assumes the vasculature conformable shape after removal of the guidewire.

48. The method of claim 47 wherein one or more aneurysm occluding devices are delivered to the aneurysm.

49. The method of claim 47 wherein the tip of the catheter is not displaced from within the lumen of the aneurysm during the delivery of the aneurysm occluding devices.

50. The method of claim 47 wherein a fill percentage of greater than 25% is achieved.

51. The method of claim 47 wherein a recurrence of the aneurysm is reduced.

52. A method of treating an aneurysm comprising:
advancing a catheter having a distal end and a proximal end and further comprising a first configuration and a second configuration wherein the first configuration is adapted and configured to be delivered through a vasculature and the second configuration is adapted and configured to assume a shape in more than one plane, in its first configuration to a target location;
positioning a distal tip of the catheter within the lumen of an aneurysm;
removing a guidewire from within the lumen of the catheter;
wherein the catheter assumes the vasculature conformable shape after removal of the guidewire.

53. The method of claim 52 wherein one or more aneurysm occluding devices are delivered to the aneurysm.

54. The method of claim 52 wherein the tip of the catheter is not displaced from within the lumen of the aneurysm during the delivery of the aneurysm occluding devices.

55. The method of claim 52 wherein a fill percentage of greater than 25% is achieved.

56. The method of claim 52 wherein a recurrence of the aneurysm is reduced.

57. A method of making a catheter comprising:
obtaining an image of a vasculature of a patient;
identifying the three-dimensional geometry of the vasculature;
manufacturing a catheter having a distal end and a proximal end and further comprising a first configuration and a second configuration wherein the first configuration is adapted and configured to be delivered through a vasculature and the second configuration is adapted and configured to assume a vasculature conformable shape;

58. The method of claim 57 wherein the method uses a plurality of vasculature images from a plurality of patients.

59. The method of claim 57 wherein the catheter is a patient specific catheter.

60. A method of making a catheter comprising:
obtaining an image of a vasculature of a patient;
identifying the three-dimensional geometry of the vasculature;
manufacturing a catheter having a distal end and a proximal end and further comprising a first configuration and a second configuration wherein the first configuration is adapted and configured to be delivered through a vasculature and the second configuration is adapted and configured to assume a shape in more than one plane;

61. The method of claim 60 wherein the method uses a plurality of vasculature images from a plurality of patients.

62. The method of claim 60 wherein the catheter is a patient specific catheter.

63. A mandrel comprising:
a distal end and a proximal end and a configuration configured to impart a vasculature conformable shape to catheter.

64. The mandrel of claim 63 wherein the configuration is determined based on a vasculature image from a patient.

65. The mandrel of claim 63 wherein the configuration is determined based on an average vasculature image from a plurality of patients.

66. The mandrel of claim 63 wherein the configuration is achieved through computer modeling of one or more vasculature images.

67. The mandrel of claim 63 wherein the mandrel has a complex curvature.

68. The mandrel of claim 63 wherein the mandrel is adapted and configured to impart a patient-specific configuration to a catheter.

69. The mandrel of claim 63 wherein the configuration is multi-planar.

70. The mandrel of claim 63 wherein the configuration further comprises three or more curves.

71. The mandrel of claim 63 wherein the configuration of the catheter is determined from an analysis of a plurality of images of patient vasculature.
